SEQUENCE LISTING (7 sequences)

SEQ ID NO: 1 - skipped

SEQ ID NO: 2 - protein, 5 aa, synthetic construct
AEKNL

SEQ ID NO: 3-4 - skipped (2 entries)

SEQ ID NO: 5 - protein, 4 aa, synthetic construct
NLKM

SEQ ID NO: 6 - protein, 4 aa, synthetic construct
MIIL

SEQ ID NO: 7 - skipped

(19) United States
(12) Patent Application Publication
(10) Pub. No.: US 2026/0200973 A1
(43) Pub. Date: Jul. 16, 2026

(54) PEPTIDE INHIBITING FORMATION OF SNARE COMPLEXES, AND COSMETIC COMPOSITION CONTAINING SAME

(71) Applicant: SUPADELIXIR INC., Chuncheon-si (KR)

(72) Inventors: Jang Hee HAHN, Chuncheon-si (KR); Min Seo KIM, Chuncheon-si (KR)

(21) Appl. No.: 18/834,598

(22) PCT Filed: Sep. 12, 2023
(86) PCT No.: PCT/KR2023/013669

§ 371 Date: Jul. 30, 2024

(30) Foreign Application Priority Data
Sep. 12, 2023 (KR) 10-2023-0121128

(51) Int. Cl. C07K 5/093; A61K 8/64; A61K 38/00; A61Q 19/08; C07K 5/083; C07K 5/09; C07K 5/103; C07K 5/113; C07K 7/06

(52) U.S. Cl. CPC C07K 5/0819; A61K 8/64; A61Q 19/08; C07K 5/0806; C07K 5/0815; C07K 5/1013; C07K 5/1021; C07K 7/06; A61K 38/00; A61K 2800/10

(57) ABSTRACT

Proposed are a peptide that exhibits efficacy similar to Botox by binding to SNAP-25 or VAMP and inhibiting the formation of SNARE complexes, and a cosmetic composition containing the same.

### TECHNICAL FIELD

The present disclosure relates to a peptide that exhibits efficacy similar to Botox by binding to SNAP-25 or VAMP and inhibiting the formation of SNARE complexes and to a cosmetic composition containing the same.

### BACKGROUND ART

SNARE complexes made of VAMP, Syntaxin, and SNAP-25 promote the secretion of acetylcholine, which is a neurotransmitter. The secreted acetylcholine causes muscles to contract, forming wrinkles. More specifically, acetylcholine moves repeatedly within the muscles, exerting continuous pressure on the skin, changing the collagen structure, and destroying elastin, finally causing muscle cells to be contracted and wrinkles to be formed.

Accordingly, technologies are being widely developed to prevent the formation of the SNARE complexes and inhibit the secretion of acetylcholine to provide a wrinkle reduction effect. An example is Botox described in the following patent document. Botox decomposes SNAP-25 and inhibits the secretion of acetylcholine, thereby inhibiting muscle contraction and providing a temporary wrinkle reduction effect.

### Patent Document

Patent document: Patent No. 10-1506204 (registered on Mar. 20, 2015), titled “Medicine Composition Reinforcing Botox Function and Comprising Fat Decomposition Function”

However, Botox has safety issues depending on the dosage, so Botox has to be administered by a specialist, which causes inconvenience in use.

### DISCLOSURE

### Technical Problem

The present disclosure was devised to solve the problems.

An objective of the present disclosure is to provide a peptide that reduces a physical binding between SNAP-25 and VAMP, inhibits the formation of the SNARE complexes, and inhibits acetylcholine secretion, thereby providing a temporary wrinkle reduction effect, and also to provide a cosmetic composition using the same.

### Technical Solution

To achieve the described objective, the present disclosure is implemented by embodiments having the following configuration.

According to one embodiment of the present disclosure, a peptide according to the present disclosure is made of any one amino acid sequence selected from ones represented by SEQ ID NOS: 1 to 7.

According to another embodiment of the present disclosure, the peptide according to the present disclosure inhibits the formation of SNARE complexes.

According to yet another embodiment of the present disclosure, the peptide according to the present disclosure is used to reduce wrinkles.

According to yet another embodiment of the present disclosure, the peptide according to the present disclosure reduces a physical binding between SNAP-25 and VAMP, inhibiting the formation of the SNARE complexes and inhibiting acetylcholine secretion, thereby providing a temporary wrinkle reduction effect.

According to yet another embodiment of the present disclosure, a cosmetic composition according to the present disclosure contains a peptide including any one amino acid sequence selected from ones represented by SEQ ID NOs: 1 to 7.

According to yet another embodiment of the present disclosure, a composition to reduce skin wrinkles according to the present disclosure contains a peptide including any one amino acid sequence selected from ones represented by SEQ ID NOs: 1 to 7.

According to yet another embodiment of the present disclosure, the cosmetic composition according to the present disclosure is used to reduce wrinkles.

### Advantageous Effects

The following effects can be achieved by the described embodiments of the present disclosure.

The present disclosure uses a peptide including any one amino acid sequence selected from ones represented by SEQ ID NOs: 1 to 7, so the peptide can produce a temporary wrinkle reduction effect similar to Botox by inhibiting the formation of the SNARE complexes.

### DESCRIPTION OF DRAWINGS

FIG. 1 is a graph showing the effects of peptides on the physical binding between SNAP-25 and VAMP according to one embodiment of the present disclosure.

### BEST MODE

Hereinafter, peptides that inhibit the formation of SNARE complexes according to the present disclosure and cosmetic compositions containing the same will be described in detail with reference to the attached drawing. Unless otherwise specified, all terms in this specification have the same general meaning as understood by those skilled in the art to which the present disclosure pertains, and when there is a conflict with the meaning of the terms used herein, this specification follows the definitions used in the specification. Additionally, detailed descriptions of well-known functions and configurations that may unnecessarily obscure the gist of the present disclosure are omitted. Throughout the specification, when a part “includes” a certain component, this means that the part may further include other components rather than excluding other components unless specifically stated to the contrary.

According to one embodiment of the present disclosure, the present disclosure relates to peptides that inhibit the formation of the SNARE complexes. The peptides are made of amino acid sequences represented by SEQ ID NO: 1 (Glu-Met-Gln), SEQ ID NO: 2 (Ala-Glu-Lys-Asn-Leu), SEQ ID NO: 3 (Glu-Lys-Asn), SEQ ID NO: 4 (Lys-Asn-Leu), SEQ ID NO: 5 (Asn-Leu-Lys-Met), SEQ ID NO: 6 (Met-Ile-Ile-Leu), or SEQ ID NO: 7 (Gly-Val-Ile). The peptides may provide a wrinkle reduction effect by inhibiting the formation of the SNARE complexes. More specifically, the peptides reduce a physical binding between SNAP-25 and VAMP, inhibiting the formation of the SNARE complexes and inhibiting acetylcholine secretion, thereby providing a temporary wrinkle reduction effect. The peptides may be used in cosmetic compositions and therapeutic compositions.

According to another embodiment, the present disclosure relates to cosmetic compositions containing a peptide including any one amino acid sequence selected from ones represented by SEQ ID NOs: 1 to 7. The cosmetic compositions may reduce skin wrinkles. Various known ingredients that may provide formulation stability and additional skin condition improvement effects may be further included in the cosmetic compositions.

Hereinafter, the present disclosure will be described in more detail through examples. However, these are only for explaining the present disclosure in more detail, and the scope of the present disclosure is not limited thereto.

### <Example 1> Synthesis of Peptides

Peptides represented by SEQ ID NOs: 1 to 7 listed in Table 1 below were synthesized by an FMOC solid-phase method using an automated synthesizer (PeptrEx-R48, Peptron, Korea). The synthesized peptides were purified and analyzed by reverse-phase high-performance liquid chromatography (Prominence LC-20AB, Shimadzu, Japan) using a C18 analytical RP column (Shiseido Capcell Pak). The synthesized peptides were identified by mass spectrometry (HP 1100 Series LC/MSD, Hewlett-Packard, USA).

TABLE 1

| Peptide name | Sequence number | Amino acid sequence |
|---|---|---|
| Botox-Pep 1 | Sequence number 1 | Glu-Met-Gln |
| Botox-Pep 2 | Sequence number 2 | Ala-Glu-Lys-Asn-Leu |
| Botox-Pep 3 | Sequence number 3 | Glu-Lys-Asn |
| Botox-Pep 4 | Sequence number 4 | Lys-Asn-Leu |
| Botox-Pep 5 | Sequence number 5 | Asn-Leu-Lys-Met |
| Botox-Pep 6 | Sequence number 6 | Met-Ile-Ile-Leu |
| Botox-Pep 7 | Sequence number 7 | Gly-Val-Ile |

### <Example 2> Confirmation of Whether Peptides of Example 1 Reduce Physical Binding Between VAMP and SNAP-25

1. Botulinum toxin A decomposes SNAP-25, which is one of the SNARE complexes' components and inhibits acetylcholine's secretion, thereby inhibiting muscle contraction and exhibiting a temporary wrinkle reduction effect. To confirm whether the peptides of Example 1 function similarly to Botox by inhibiting the binding between SNAP-25, which is a component protein of the SNARE complexes, to VAMP, the effects of the peptides on the physical binding between SNAP-25 and VAMP were evaluated using an in-situ PLA (proximity ligation assay) method.

2. Specifically, 4.5×10⁴ SH-SY5Y cells (human neuroblasts) were seeded into each well of a 24-well microplate with MEM serum medium. The cells were cultured in an incubator at a temperature of 37° C. and with 5% CO₂ for 24 hours. Next, the cells were treated with each of the peptides of Example 1, and the cells were incubated under the same conditions for 24 hours. The concentration of each peptide in the medium was set to 10 μM. The control group received no treatment. Afterward, the cells in each well were washed with PBS and fixed by treatment with 2% formaldehyde for 15 minutes, and then the cells were treated with 0.1% TritonX-100 for 5 minutes to increase antibody permeability into the cells. Anti-VAMP-1/2/3 polyclonal antibodies (Santa Cruz, USA) and anti-SNAP-25 monoclonal antibodies (Santa Cruz, USA) were added, and PLA probes were applied using an in-situ PLA kit (Sigma-Aldrich). Subsequently, hybridization, ligation, amplification, and mounting steps were performed.

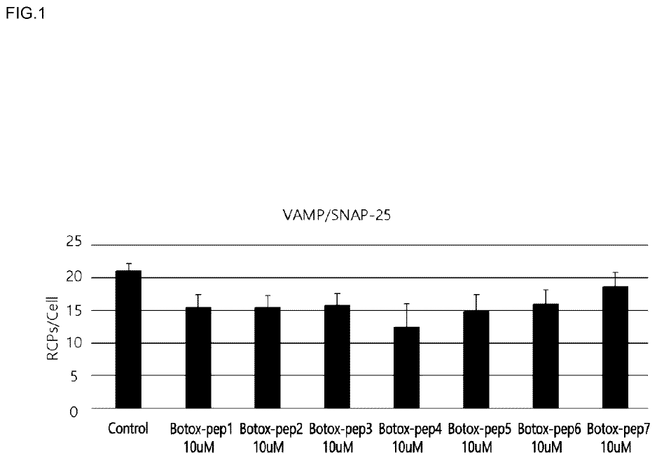

3. Afterward, the physical interaction of SNAP-25 and VAMP antibodies was quantified by measuring luminescence signals (PLA signals) detected in each cell using a fluorescence microscope (Logosbio/CelenaS, Korea), and the results are shown in FIG. 1.

4. As seen in FIG. 1, when treated with Botox-Pep 1 to 7 of Example 1, the physical binding between SNAP-25 and VAMP increased compared to the untreated control group. That is, Botox-Pep 1 to 7 helped temporarily reduce skin wrinkles by inhibiting muscle contraction by reducing the physical binding between SNAP-25 and VAMP.

### <Example 3> Evaluation of Whether Peptides of Example 1 have Effects on Acetylcholine Secretion of Nerve Cells

1. When the SNARE complexes are formed, the nerve transmitter acetylcholine is secreted and exported to the muscles, causing wrinkles. To confirm whether the peptides of Example 1 inhibit acetylcholine secretion from nerve cells, acetylcholine secretion was measured using an Acetylcholine assay kit.

2. Specifically, SH-SY5Y cells were prepared to have a concentration of 1×10⁶ per well and then treated with each of the peptides of Example 1. The cells were incubated under the same conditions for 24 hours. The concentration of each peptide in the medium w was set to 10 μM. The control group received no treatment. Next, the SH-SY5Y cells were washed three times with PBS, and then the cells were lysed using an Acetylcholine assay kit (Abcam, USA). Afterward, a reaction solution containing the choline probes was applied, and the amount of intracellular acetylcholine was measured using a microplate reader. Using the amount of acetylcholine in the control group as a standard (100%), the relative amount of acetylcholine in the experimental group was calculated and is shown in Table 2.

3. As seen in Table 2, when treated with Botox-Pep 1 to 7 of Example 1, the amount of intracellular acetylcholine was reduced. It may be inferred that the peptides prepared in Example 1 inhibited the formation of the SNARE complexes that cause acetylcholine to be secreted. In addition, it may be inferred that the peptides might provide a temporary wrinkle reduction effect since the secretion of acetylcholine was inhibited.

TABLE 2

| Botox-pep | Untreated | 1 | 2 | 3 | 4 | 5 | 6 | 7 |
|---|---|---|---|---|---|---|---|---|
| Acetylcholine | 100 | 81 | 80 | 82 | 78 | 88 | 80 | 93 |
| release | | | | | | | | |
| (% of control) | | | | | | | | |

### <Example 4> Confirmation of Whether Cosmetic Compositions Containing Peptides of Example 1 have Effects on Wrinkle Reduction

1. Cosmetic compositions were prepared by adding each of the peptides prepared in Example 1 to a commercially available solubilized ampoule base at a concentration of 50 ppm.

2. Nine adults in their 20s to 60s were asked to apply the same amount of each of the cosmetic compositions to the wrinkles under their eyes every morning and evening for 2 weeks. Afterward, the degree of wrinkles under the eyes before and after use was evaluated using a skin diagnosis device (JNAUS, Korea). The results are shown in Table 3. Argireline® peptide (Lubrizol, USA), a known Botox ingredient, was used as a control group.

3. As seen in Table 3, the cosmetic compositions containing each of the peptides prepared in Example 1 had an effect of reducing wrinkles on the skin under the eyes. That is, Botox-Pep 1 to 7 might be used for cosmetic compositions to help temporarily reduce wrinkles.

TABLE 3

| Cosmetic | Control | Botox-pep | | | | | | |
|---|---|---|---|---|---|---|---|---|
| composition | group | 1 | 2 | 3 | 4 | 5 | 6 | 7 |
| Wrinkle | 6.7 | 13.7 | 14.6 | 12.1 | 18.6 | 13.3 | 14.1 | 7.9 |
| reduction | | | | | | | | |
| rate under | | | | | | | | |
| the eyes | | | | | | | | |
| (%) | | | | | | | | |

Herein above, the applicants have described preferred examples of the present disclosure, but such examples are only examples that implement the technical idea of the present disclosure. Any changes or modifications should be construed as falling within the scope of the present disclosure as long as the changes or modifications embody the technical idea of the present disclosure.

1-4. (canceled)

5. A cosmetic composition comprising a peptide consisting of any one amino acid sequence selected from ones represented by SEQ ID NOs: 1 to 7.

6-7. (canceled)

8. A synthetic peptide consisting of any one amino acid sequence selected from ones represented by SEQ ID NOs: 1 to 7, wherein the synthetic peptide is a chemically synthesized peptide.

9. A peptide having an activity of reducing a physical binding between SNAP-25 and VAMP, consisting of any one amino acid sequence selected from ones represented by SEQ ID NOs: 1 to 7.

10. A pharmaceutical composition comprising a peptide consisting of any one amino acid sequence selected from ones represented by SEQ ID NOs: 1 to 7.

11. A method for reducing a skin wrinkle, comprising applying an effective amount of a peptide consisting of any one amino acid sequence selected from ones represented by SEQ ID NOs: 1 to 7 to the skin of a subject in need thereof.

12. The method of claim 11, wherein the peptide reduces a physical binding between SNAP-25 and VAMP, inhibiting the formation of a SNARE complex, and inhibiting acetylcholine secretion, thereby providing a temporary skin wrinkle reduction effect.
